(19) United States
(12) Patent Application Publication
(10) Pub. No.: US 2025/0057504 A1
(43) Pub. Date: Feb. 20, 2025

(54) Intravascular Ultrasound Catheter

(71) Applicant: Boston Scientific Scimed, Inc., Maple Grove, MN (US)

(72) Inventors: Cass Alexander Hanson, St. Paul, MN (US); Joel M. WasDyke, Independence, MN (US); Paul F. Chouinard, Maple Grove, MN (US)

(73) Assignee: Boston Scientific Scimed, Inc., Maple Grove, MN (US)

(21) Appl. No.: 18/802,560

(22) Filed: Aug. 13, 2024

Related U.S. Application Data
(60) Provisional application No. 63/533,234, filed on Aug. 17, 2023.

(51) Int. Cl. A61B 8/12; A61B 8/08

(52) U.S. Cl. CPC A61B 8/12; A61B 8/0891

(57) ABSTRACT

Intravascular imaging catheters and methods for making and using intravascular imaging catheters are disclosed. An example intravascular imaging catheter may include an elongate catheter shaft having a distal end region and a proximal region. A guidewire lumen may be defined in the elongate catheter shaft. The distal end region may include an imaging window defined by an open gap in the elongate catheter shaft. An imaging core may be disposed within the elongate catheter shaft. The imaging core may include an imaging device configured to be axially aligned with the imaging window.

### CROSS-REFERENCE TO RELATED APPLICATIONS

This application claims the benefit of priority under 35 U.S.C. § 119 of U.S. Provisional Application No. 63/533,234, filed Aug. 17, 2023, the entire disclosure of which is hereby incorporated by reference.

### TECHNICAL FIELD

The present disclosure pertains to medical devices, and methods for manufacturing medical devices. More particularly, the present disclosure pertains to imaging devices such as intravascular ultrasound catheters.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An intravascular imaging catheter is disclosed. The intravascular imaging catheter comprises: an elongate catheter shaft having a distal end region and a proximal region; wherein a guidewire lumen is defined in the elongate catheter shaft; wherein the distal end region includes an imaging window defined by an open gap in the elongate catheter shaft; an imaging core disposed within the elongate catheter shaft; and wherein the imaging core includes an imaging device configured to be axially aligned with the imaging window.

Alternatively or additionally to any of the embodiments above, the distal end region is connected to the proximal region by a bridge region of the elongate catheter shaft.

Alternatively or additionally to any of the embodiments above, the guidewire lumen is defined by a guidewire lumen shaft extending from the proximal region of the elongate catheter shaft to the distal end region of the elongate catheter shaft.

Alternatively or additionally to any of the embodiments above, the distal end region is coupled to the proximal region by the guidewire lumen shaft.

Alternatively or additionally to any of the embodiments above, the open gap is formed by a cutout in the elongate catheter shaft.

Alternatively or additionally to any of the embodiments above, the distal end region of the elongate catheter shaft has a proximal end, wherein the proximal region of the elongate catheter shaft has a distal end, and wherein the open gap is disposed between the proximal end of the distal end region and the distal end of the proximal region.

Alternatively or additionally to any of the embodiments above, the imaging core is configured to shift between a delivery position where the imaging device is disposed within the distal end region and an imaging position where the imaging device is aligned with the imaging window.

Alternatively or additionally to any of the embodiments above, shifting the imaging core from the delivery position to the imaging position includes axially shifting the imaging core relative to the elongate catheter shaft.

Alternatively or additionally to any of the embodiments above, the imaging device is coupled to an imaging housing.

Alternatively or additionally to any of the embodiments above, the imaging housing has a proximal section that extends into the proximal region of the elongate catheter shaft.

Alternatively or additionally to any of the embodiments above, the imaging housing has a distal section that extends into the distal end region of the elongate catheter shaft.

Alternatively or additionally to any of the embodiments above, the imaging device includes an ultrasound transducer.

An intravascular imaging catheter is disclosed. The intravascular imaging catheter comprises: an elongate imaging catheter sheath having a distal end region, a proximal region, an imaging window region disposed between the distal end region and the proximal region; wherein the imaging window region is defined by an opening in the elongate imaging catheter sheath; a guidewire lumen shaft extending from the proximal region of the elongate imaging catheter sheath to the distal end region of the elongate imaging catheter sheath; an imaging core disposed within the elongate imaging catheter sheath; wherein the imaging core includes an ultrasound imaging device; and wherein the imaging core is configured to shift between a delivery position where the ultrasound imaging device is disposed within the distal end region and an imaging position where the ultrasound imaging device is aligned with the imaging window region.

Alternatively or additionally to any of the embodiments above, the distal end region of the elongate imaging catheter sheath is coupled to the proximal region of the elongate imaging catheter sheath by the guidewire lumen shaft.

Alternatively or additionally to any of the embodiments above, the opening is formed by a cutout in the elongate imaging catheter sheath.

Alternatively or additionally to any of the embodiments above, the distal end region of the elongate imaging catheter sheath has a proximal end, wherein the proximal region of the elongate imaging catheter sheath has a distal end, and wherein the opening is disposed between the proximal end of the distal end region and the distal end of the proximal region.

Alternatively or additionally to any of the embodiments above, shifting the imaging core from the delivery position to the imaging position includes axially shifting the imaging core relative to the elongate imaging catheter sheath.

A method for imaging a vascular region is disclosed. The method comprises: advancing an intravascular imaging catheter through a blood vessel to a position adjacent to an area of interest; wherein the intravascular imaging catheter comprises: an elongate catheter shaft having a distal end region and a proximal region, wherein a guidewire lumen is defined in the elongate catheter shaft, wherein the distal end region includes an imaging window defined by an open gap in the elongate catheter shaft, an imaging core disposed within the elongate catheter shaft, and wherein the imaging core includes an imaging device; aligning the imaging device with the imaging window; and imaging the blood vessel using the imaging device and while proximally retracting the elongate catheter shaft.

Alternatively or additionally to any of the embodiments above, the imaging core is configured to shift between a delivery position where the imaging device is disposed within the distal end region and an imaging position where the imaging device is aligned with the imaging window; and wherein aligning the imaging device with the imaging window includes shifting the imaging core from the delivery position to the imaging position.

Alternatively or additionally to any of the embodiments above, imaging the blood vessel using the imaging device and while proximally retracting the elongate catheter shaft includes proximally retracting the elongate catheter shaft and the imaging core.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of a portion of an example medical device.

FIG. 2 is a side view of a portion of an example medical device.

FIG. 3 is a side view of a portion of an example medical device.

FIG. 4 is a side view of a portion of an example medical device.

FIG. 5 is a side view of a portion of an example medical device.

FIG. 6 is a side view of a portion of an example medical device.

FIG. 7 is a side view of a portion of an example medical device.

FIG. 8 is a side view of a portion of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

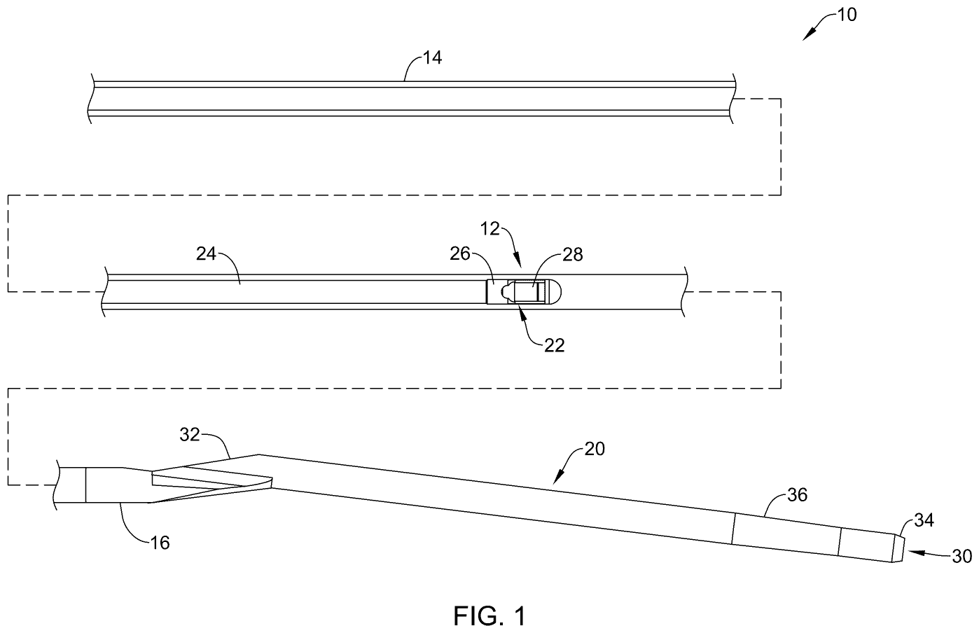

FIG. 1 is a side view of a portion of an example medical device 10. In at least some instances, the medical device 10 takes the form of an imaging medical device. For example, the medical device 10 may be an intravascular ultrasound (IVUS) device that may be used to image a blood vessel. In some of these and in other instances the medical device may be an optical coherence tomography (OCT) imaging device, a near-infrared spectroscopy (NIRS) imaging device, near-infrared fluorescence (NIRF) imaging device, a photoacoustic imaging device, a fluorescence-lifetime imaging device, combinations thereof (including combinations that include IVUS), and/or the like. In addition to be used for intravascular imaging, the medical device 10 may also be used for pulmonary procedures/imaging. The structure/form of the medical device 10 can vary. In some instances, the medical device 10 may include an elongate catheter shaft 12 having a proximal end region 14 and a distal end region 16. A tip member 20 may be coupled to or otherwise disposed adjacent to the distal end region 16. The tip member 20 may include a guidewire lumen 30 having a guidewire exit port 32, an atraumatic distal end 34, one or more radiopaque markers 36, and/or other features. In some embodiments, the tip member 20 may extend at a non-parallel angle to the proximal end region 14 of the elongate catheter shaft 12.

An imaging assembly 22 (e.g., which may sometime be referred to as an imaging core) may be disposed within a lumen of the elongate catheter shaft 12. In general, the imaging core 22 may be used to capture/generate images of a blood vessel. In some instances, the medical device may include devices and/or features similar to those disclosed in U.S. Patent Application Pub. No. US 2012/0059241 and U.S. Patent Application Pub. No. US 2017/0164925, the entire disclosures of which are herein incorporated by reference. In at least some instances, the medical device 10 may resemble and/or include features that resemble the OPTICROSS™ Imaging Catheter, commercially available from BOSTON SCIENTIFIC, Marlborough, MA.

The imaging core 22 may include a drive shaft or cable 24, a housing 26, and an imaging member or transducer 28 coupled to the drive shaft 24 and/or housing 26. In at least some instances, the transducer 28 includes an ultrasound transducer. Other transducers are also contemplated. The transducer 28 may be rotatable and/or axially translatable relative to the elongate catheter shaft 12. For example, the drive shaft 24 may be rotated and/or translated in order to rotate and/or translate the transducer 28 (and the housing 26).

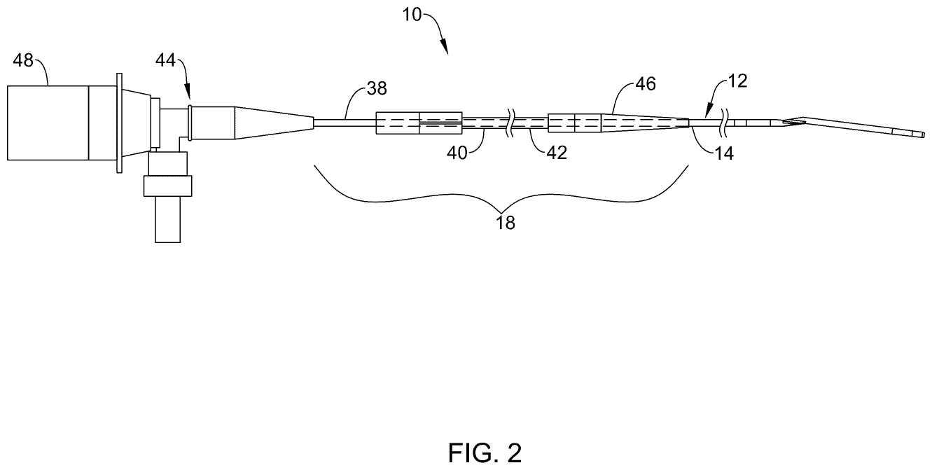

In some instances, the proximal end region 14 of the elongate catheter shaft 12 may be coupled to a telescoping assembly 18 as shown in FIG. 2. In general, the telescoping assembly 18 may be configured to allow the medical device operator to move the drive shaft 24 including the imaging core 22 proximally and distally within the catheter (e.g., relative to the elongate catheter shaft 12), without having to move the entire catheter within the patient. This allows the catheter operator to easily change the location of the imaging core 22 within the patient. For example, the telescoping assembly 18 may be actuated to change the location of the imaging core 22 within the elongate catheter shaft 12.

The proximal end region 14 of the elongate catheter shaft 12 may be coupled to the telescoping assembly 18. For example, the proximal end region 14 of the elongate catheter shaft 12 may be coupled to a distal hub 46 of the telescoping assembly 18. A proximal hub 44 may be coupled to the telescoping assembly 18 (e.g., at the distal end of the telescoping assembly 18). The drive shaft 24 (see FIG. 1) may extend through the telescoping assembly 18 and be coupled to and/or otherwise secured to the proximal hub 44. The proximal hub 44 may include a connector assembly 48. In general, the connector assembly 48 may allow the medical device 10 (e.g., the elongate catheter shaft 12) to attached to a control unit (e.g., a motor drive unit and/or the like) as described in more detail herein.

The telescoping assembly 18 may include a first sheath 38 and a second sheath 40. In some instances, the first sheath 38 may be understood to be an inner telescoping tube 38 and the second sheath 40 may be understood to be an outer telescoping tube 40. Generally, the outer telescoping tube 40 may be disposed over the inner telescoping tube 38. The inner telescoping tube 38 may be coupled to or otherwise secured to the proximal hub 44. The outer telescoping tube 40 may be coupled or otherwise secured to the distal hub 46. The inner telescoping tube 38 may be axially and/or rotatably moveable relative to the outer telescoping tube 40. Because the drive shaft 24 may be secured to the proximal hub 44 and/or the inner telescoping tube 38 and because the elongate catheter shaft 12 may be secured to the distal hub 46, movement of the proximal hub 44 relative to the distal hub 46 results in movement of the inner telescoping tube 38 and the drive shaft 24 relative to the distal hub 46 and/or the elongate catheter shaft 12.

Flushing the medical device 10, for example to remove air and/or bubbles that could be trapped in the medical device 10, may include flushing the telescoping assembly 18 and/or the distal end region 16 of the elongate catheter shaft 12. It may be desirable to simplify the flushing processes. For example, it may be desirable to reduce the number of structures that need to be flushed. Disclosed herein are alternative medical devices that include structural modifications that may, for example, help to simplify flushing of the intravascular imaging catheter. Some of these and other benefits of the alternative medical devices are disclosed herein.

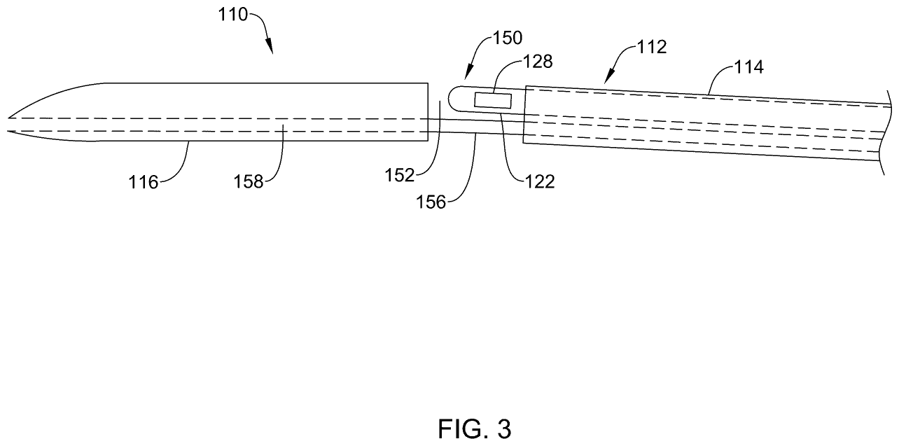

FIG. 3 illustrates another example medical device 110 that may be similar in form and function to other medical device disclosed herein. In some instances, the medical device 110 may be an intravascular imaging device 110. The intravascular imaging device 110 may include an elongate catheter shaft or imaging sheath 112. The elongate catheter shaft 112 may include a proximal region 114 and a distal end region 116. It can be appreciated that the terms “elongate” and/or “elongated” are relative terms. The actual length of the elongate catheter shaft 112 and the regions/sections thereof can vary. For example, some portions of and/or the entire length of the elongate catheter shaft 112 can be relatively short in some instances.

An imaging window 150 may be defined in the elongate catheter shaft 112. In some instances, the imaging window 150 may be formed or defined by an open gap 152 in the elongate catheter shaft 112. In some instances, the open gap 152 is formed by a cutout in the elongate catheter shaft 112. The cutout may separate the proximal region 114 and the distal end region 116. For example, the distal end region 116 of the elongate catheter shaft 112 may have a proximal end, the proximal region 114 of the elongate catheter shaft 112 has a distal end, and the open gap 152 is disposed between the proximal end of the distal end region 116 and the distal end of the proximal region 114. In other words, the proximal region 114 and the distal end region 116 may be axially spaced apart from one another and the open gap 152 is defined at least in part by the space.

An imaging core 122 may be disposed within the elongate catheter shaft 112. The imaging core 122 may include an imaging device 128. In at least some instances, the imaging device 128 may include an ultrasound imaging device (e.g., an ultrasound transducer). Other imaging devices are contemplated. For example, the imaging device 128 may include an OCT device, a NIRS device, a NIRF device, and/or combinations thereof (e.g., including combinations that may include an IVUS imaging device).

In some instances, the imaging core 122 may be disposed within the elongate catheter shaft 112 in a manner that substantially aligns the imaging device 128 with the imaging window 150 (and/or the open gap 152). This may be desirable for a number of reasons. For example, by disposing the imaging device 128 within an open area of the elongate catheter shaft 112, flushing of the intravascular imaging device 110 may be reduced and/or simplified. In addition, by disposing the imaging device 128 within an open area of the elongate catheter shaft 112, the imaging device 128 and/or imaging core 122 may be axially fixed relative to the elongate catheter shaft 112. Because of this, a telescoping structure (e.g., similar to the telescoping assembly 18) may not be necessary. Thus, the construction and use of the intravascular imaging device 110 may be simplified.

In some instances, the intravascular imaging device 110 may be navigated through the vasculature with the imaging device 128 with the imaging window 150 and/or the open gap 152 (e.g., as depicted in FIG. 3). Imaging may include using the imaging device 128 to generate intravascular images. This may include rotating the imaging device 128 and/or proximally retracting the imaging device 128 through the vasculature (e.g., a pullback procedure). Rotating and/or retracting the imaging device 128 may include rotating and/or proximally retracting the imaging core 122. In at least some instances, the elongate catheter shaft 112 may be rotated and/or retracted along with the imaging core 122.

In instances where the elongate catheter shaft 112 is rotated along with the imaging core 122, the imaging device 128 may be oriented in a constant orientation, which may reduce imaging of a guidewire extending through the elongate catheter shaft 112 (e.g., thereby reducing guidewire artifacts in the resultant intravascular images.).

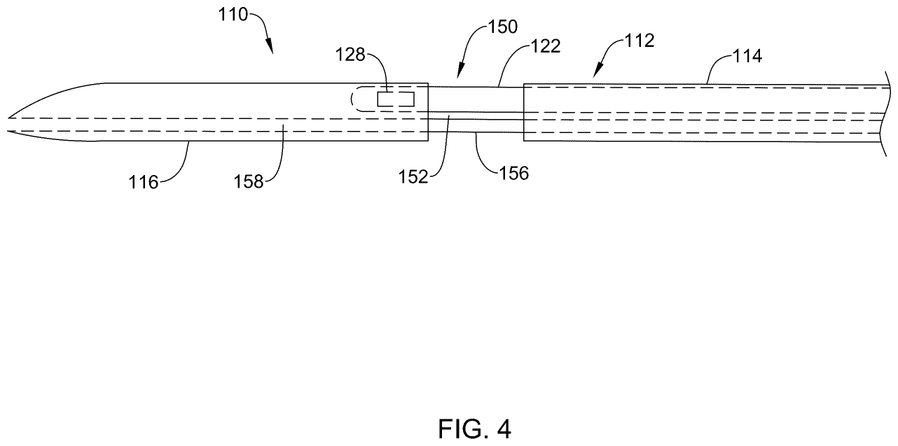

In some instances, the imaging core 122 may be configured to shift between a delivery position (e.g., as shown in FIG. 4) where the imaging device 128 is disposed within the distal end region 116 and an imaging position (e.g., as shown in FIG. 3) where the imaging device 128 is aligned with the imaging window 150. Shifting the imaging core 122 from the delivery position to the imaging position includes axially shifting the imaging core 122 relative to the elongate catheter shaft 112. This may include axially aligning the imaging device 128 with the imaging window 150 (and/or the open gap 152). For example, the imaging core 122 may be delivered with the imaging core 122 in the delivery position. When in the delivery position, the imaging device 128 is disposed within the distal end region 116. Upon reaching a target site, the imaging core 122 may be shifted to the imaging position where the imaging device 128 is disposed within and/or aligned with the imaging window 150.

A guidewire lumen shaft 156 may extend between the proximal region 114 and the distal end region 116. The guidewire lumen shaft 156 may define the guidewire lumen 158. The guidewire lumen shaft 156 may help to secure and/or bond the proximal region 114 with the distal end region 116. For example, the guidewire lumen shaft 156 may be bonded to both the proximal region 114 and the distal end region 116. In this example, the guidewire lumen shaft 156 may be separate and distinct shaft from the elongate catheter shaft 112. In other instances, the guidewire lumen shaft 156 may be integral with the elongate catheter shaft 112 and/or the guidewire lumen 158 may be formed within the elongate catheter shaft 112 (e.g., such that a separate guidewire lumen shaft 156 may not be necessary).

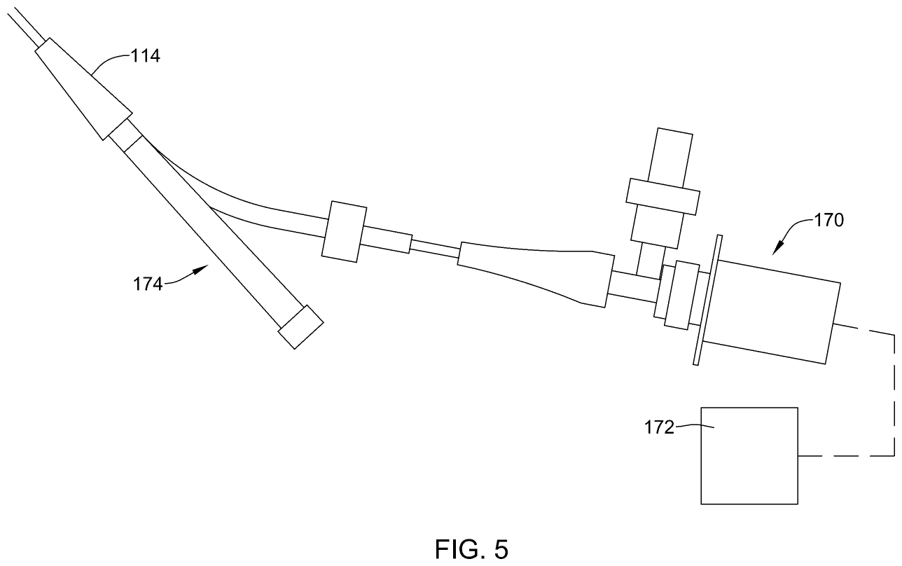

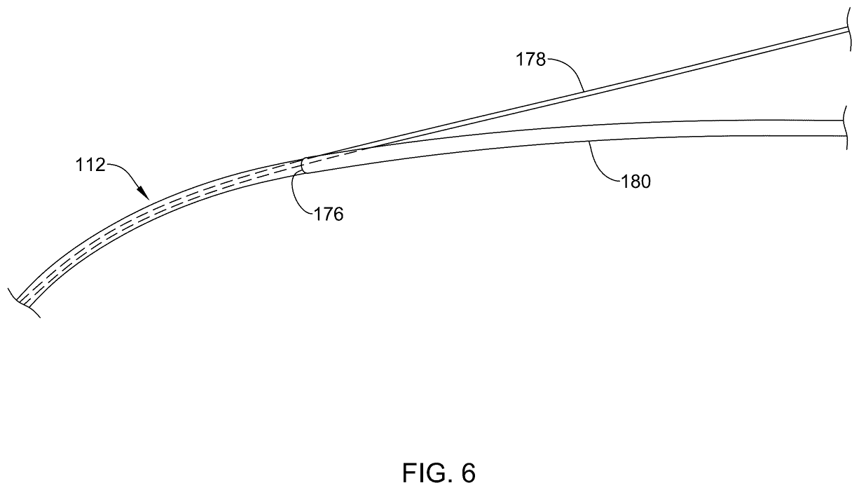

The proximal region 114 may include a proximal hub 170 configured to be coupled to a control and/or motor drive unit 172 as schematically depicted in FIG. 5. A guidewire hub 174 may be disposed adjacent to the proximal hub. In other instances, the proximal region 114 may include a single-operator-exchange or monorail port 176 as shown in FIG. 6. The monorail port 176 may allow a guidewire 178 to exit the elongate catheter shaft 112. A proximal extension region 180 of the elongate catheter shaft 112 may extend to the proximal hub 170 (e.g., the proximal hub 170 as shown in FIG. 5).

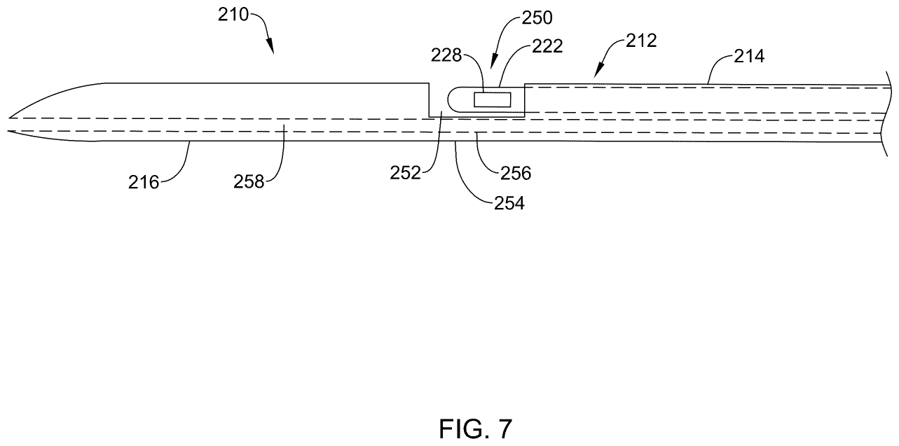

It can be appreciated that the rather than the guidewire lumen shaft 156 securing the proximal region 114 with the distal end region 116, a portion of the elongate catheter shaft 112 itself may remain and extend between the proximal region 114 and the distal end region 116. For example, FIG. 7 illustrates another example medical device 210 that may be similar in form and function to other medical devices disclosed herein. In some instances, the medical device 210 may be an intravascular imaging device 210. The intravascular imaging device 210 may include an elongate catheter shaft or imaging sheath 212. The elongate catheter shaft 212 may include a proximal region 214 and a distal end region 216. An imaging window 250 may be defined in the elongate catheter shaft 212. In some instances, the imaging window 250 may be formed or defined by an open gap 252 in the elongate catheter shaft 212. An imaging core 222 may be disposed within the elongate catheter shaft 212. The imaging core 222 may include an imaging device 228. In at least some instances, the imaging device 228 may include an ultrasound imaging device (e.g., an ultrasound transducer).

In this example, a bridge region 254 of the elongate catheter shaft 212 may extend between the proximal region 214 and the distal end region 216 of the elongate catheter shaft 212. A guidewire lumen shaft region 256 may be defined in the catheter shaft 212 that defines a guidewire lumen 258. In this example, the guidewire lumen shaft region 256 may be understood to be a region of the catheter shaft 212 that defines the guidewire lumen 258 therein. When forming the imaging window 250 in the catheter shaft 212, a portion of the catheter shaft 212 may be removed, leaving behind the bridge region 254.

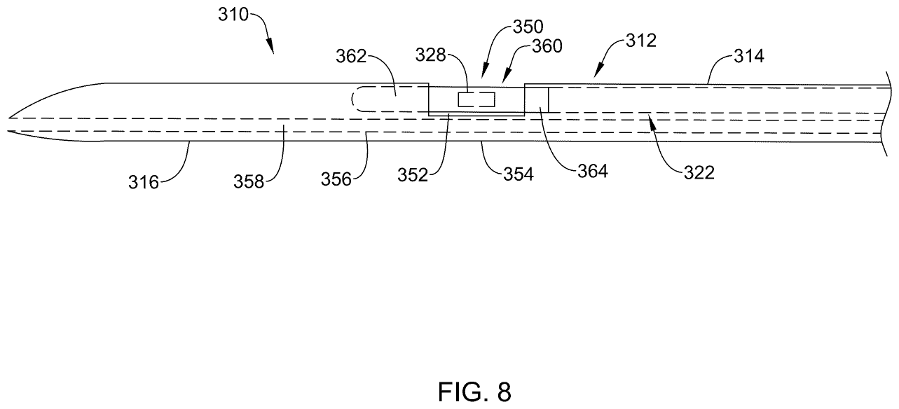

FIG. 8 illustrates another example medical device 310 that may be similar in form and function to other medical devices disclosed herein. In some instances, the medical device 310 may be an intravascular imaging device 310. The intravascular imaging device 310 may include an elongate catheter shaft or imaging sheath 312. The elongate catheter shaft 312 may include a proximal region 314 and a distal end region 316. An imaging window 350 may be defined in the elongate catheter shaft 312. In some instances, the imaging window 350 may be formed or defined by an open gap 352 in the elongate catheter shaft 312.

An imaging core 322 may be disposed within the elongate catheter shaft 312. The imaging core 322 may include an imaging device 328. In at least some instances, the imaging device 328 may include an ultrasound imaging device (e.g., an ultrasound transducer). In this example, the imaging core 322 may include an imaging housing 360. The imaging device 328 may be disposed along or otherwise coupled to the imaging housing 360. The imaging housing 360 may include a distal section 362 and a proximal section 364. The distal section 362 may extend into the distal end region 316 of the elongate catheter shaft 312. The proximal section 364 may extend into the proximal region 314 of the elongate catheter shaft 312.

In this example, a bridge region 354 of the elongate catheter shaft 312 may extend between the proximal region 314 and the distal end region 316 of the elongate catheter shaft 312. A guidewire lumen shaft region 356 may be defined in the catheter shaft 312 that defines a guidewire lumen 358. In this example, the guidewire lumen shaft region 356 may be understood to be a region of the catheter shaft 312 that defines the guidewire lumen 358 therein. When forming the imaging window 350 in the catheter shaft 312, a portion of the catheter shaft 312 may be removed, leaving behind the bridge region 354. It can be appreciated that in alternative instances, the catheter shaft 312 may be similar the catheter shaft 112 (e.g., an includes a guidewire lumen shaft similar to the guidewire lumen shaft 156).

The materials that can be used for the various components of the medical device 10 (and/or other medical devices disclosed herein) may include those commonly associated with medical devices. For simplicity purposes, the following discussion makes reference to the elongate catheter shaft 12 and other components of the medical device 10. However, this is not intended to limit the devices and methods described herein, as the discussion may be applied to other shafts and/or catheters disclosed herein.

The elongate catheter shaft 12 and/or other components of the medical device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester clastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 to achieve the same result.

In some embodiments, a degree of Magnetic Resonance Imaging (MRI) compatibility is imparted into the medical device 10. For example, the medical device 10, or portions thereof, may be made of a material that does not substantially distort the image and create substantial artifacts (e.g., gaps in the image). Certain ferromagnetic materials, for example, may not be suitable because they may create artifacts in an MRI image. The medical device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An intravascular imaging catheter, comprising:
an elongate catheter shaft having a distal end region and a proximal region;
wherein a guidewire lumen is defined in the elongate catheter shaft;
wherein the distal end region includes an imaging window defined by an open gap in the elongate catheter shaft;
an imaging core disposed within the elongate catheter shaft; and
wherein the imaging core includes an imaging device configured to be axially aligned with the imaging window.

2. The intravascular imaging catheter of claim 1, wherein the distal end region is connected to the proximal region by a bridge region of the elongate catheter shaft.

3. The intravascular imaging catheter of claim 1, wherein the guidewire lumen is defined by a guidewire lumen shaft extending from the proximal region of the elongate catheter shaft to the distal end region of the elongate catheter shaft.

4. The intravascular imaging catheter of claim 3, wherein the distal end region is coupled to the proximal region by the guidewire lumen shaft.

5. The intravascular imaging catheter of claim 1, wherein the open gap is formed by a cutout in the elongate catheter shaft.

6. The intravascular imaging catheter of claim 1, wherein the distal end region of the elongate catheter shaft has a proximal end, wherein the proximal region of the elongate catheter shaft has a distal end, and wherein the open gap is disposed between the proximal end of the distal end region and the distal end of the proximal region.

7. The intravascular imaging catheter of claim 1, wherein the imaging core is configured to shift between a delivery position where the imaging device is disposed within the distal end region and an imaging position where the imaging device is aligned with the imaging window.

8. The intravascular imaging catheter of claim 7, wherein shifting the imaging core from the delivery position to the imaging position includes axially shifting the imaging core relative to the elongate catheter shaft.

9. The intravascular imaging catheter of claim 1, wherein the imaging device is coupled to an imaging housing.

10. The intravascular imaging catheter of claim 9, wherein the imaging housing has a proximal section that extends into the proximal region of the elongate catheter shaft.

11. The intravascular imaging catheter of claim 9, wherein the imaging housing has a distal section that extends into the distal end region of the elongate catheter shaft.

12. The intravascular imaging catheter of claim 1, wherein the imaging device includes an ultrasound transducer.

13. An intravascular imaging catheter, comprising:
an elongate imaging catheter sheath having a distal end region, a proximal region, an imaging window region disposed between the distal end region and the proximal region;
wherein the imaging window region is defined by an opening in the elongate imaging catheter sheath;
a guidewire lumen shaft extending from the proximal region of the elongate imaging catheter sheath to the distal end region of the elongate imaging catheter sheath;
an imaging core disposed within the elongate imaging catheter sheath;
wherein the imaging core includes an ultrasound imaging device; and
wherein the imaging core is configured to shift between a delivery position where the ultrasound imaging device is disposed within the distal end region and an imaging position where the ultrasound imaging device is aligned with the imaging window region.

14. The intravascular imaging catheter of claim 13, wherein the distal end region of the elongate imaging catheter sheath is coupled to the proximal region of the elongate imaging catheter sheath by the guidewire lumen shaft.

15. The intravascular imaging catheter of claim 13, wherein the opening is formed by a cutout in the elongate imaging catheter sheath.

16. The intravascular imaging catheter of claim 13, wherein the distal end region of the elongate imaging catheter sheath has a proximal end, wherein the proximal region of the elongate imaging catheter sheath has a distal end, and wherein the opening is disposed between the proximal end of the distal end region and the distal end of the proximal region.

17. The intravascular imaging catheter of claim 13, wherein shifting the imaging core from the delivery position to the imaging position includes axially shifting the imaging core relative to the elongate imaging catheter sheath.

18. A method for imaging a vascular region, the method comprising:
advancing an intravascular imaging catheter through a blood vessel to a position adjacent to an area of interest;
wherein the intravascular imaging catheter comprises:
an elongate catheter shaft having a distal end region and a proximal region,
wherein a guidewire lumen is defined in the elongate catheter shaft,
wherein the distal end region includes an imaging window defined by an open gap in the elongate catheter shaft,
an imaging core disposed within the elongate catheter shaft, and
wherein the imaging core includes an imaging device;
aligning the imaging device with the imaging window; and
imaging the blood vessel using the imaging device and while proximally retracting the elongate catheter shaft.

19. The method of claim 18, wherein the imaging core is configured to shift between a delivery position where the imaging device is disposed within the distal end region and an imaging position where the imaging device is aligned with the imaging window; and wherein aligning the imaging device with the imaging window includes shifting the imaging core from the delivery position to the imaging position.

20. The method of claim 18, wherein imaging the blood vessel using the imaging device and while proximally retracting the elongate catheter shaft includes proximally retracting the elongate catheter shaft and the imaging core.
